(19) United States
(12) Patent Application Publication
(10) Pub. No.: US 2023/0404075 A1
(43) Pub. Date: Dec. 21, 2023

(54) RHIZOBIA SYMBIOSIS PROMOTING AGENT AND SYMBIOSIS PROMOTION METHOD

(71) Applicants: Satoshi KONDO, Miyoshi-shi, Aichi (JP); Madoka ABE, Nagoya-shi, Aichi (JP); Yasuyo SHIMAMOTO, Nagakute-shi, Aichi (JP); Naoya TAKEDA, Sanda-shi, Hyogo (JP); Akira AKAMATSU, Sanda-shi, Hyogo (JP); Atsushi NAGANO, Otsu-shi, Shiga (JP)

(72) Inventors: Satoshi KONDO, Miyoshi-shi, Aichi (JP); Madoka ABE, Nagoya-shi, Aichi (JP); Yasuyo SHIMAMOTO, Nagakute-shi, Aichi (JP); Naoya TAKEDA, Sanda-shi, Hyogo (JP); Akira AKAMATSU, Sanda-shi, Hyogo (JP); Atsushi NAGANO, Otsu-shi, Shiga (JP)

(73) Assignees: TOYOTA JIDOSHA KABUSHIKI KAISHA, Toyota-shi, Aichi (JP); KWANSEI GAKUIN EDUCATIONSL FOUNDATION, Nishinomiya-shi, Hyogo (JP); RYUKOKU UNIVERSITY, Kyoto-shi, Kyoto (JP)

(21) Appl. No.: 18/036,739

(22) PCT Filed: Nov. 16, 2021
(86) PCT No.: PCT/JP2021/042115

§ 371 Date: May 12, 2023

(30) Foreign Application Priority Data
Nov. 16, 2020 (JP) 2020-190053

(51) Int. Cl. A01N 37/38; A01P 21/00; A01H 3/04; A01N 37/10

(52) U.S. Cl. CPC A01N 37/38; A01P 21/00; A01H 3/04; A01N 37/10

(57) ABSTRACT

Provided is an agent for promoting root nodule formation, which contains cinnamic acid or hydroxycinnamic acid as an active ingredient.

### TECHNICAL FIELD

The present invention relates to a symbiosis-promoting agent and a symbiosis-promoting method for promoting symbiosis with a plant by rhizobia living symbiotically with the roots of the plant, and a plant cultivation method.

### BACKGROUND ART

Many leguminous plants form granular root nodules in their roots, and rhizobia (bacteria) coexist in these root nodules to fix nitrogen from the air as a nutrient. In addition to leguminous plants, other plants such as alder (Alnus japonica), autumn olive (Elaeagnus umbellata), cherry silverberry (Elaeagnus multiflora), Chinese bayberry (Myrica rubra), Yachiyanagi (Myrica gale L. var. tomentosa C.DC.), and Dokuutsugi (Coriaria japonica) are known as plants in which rhizobia coexist. In other words, rhizobia, a type of soil bacteria, invade the roots of plants to form root nodules to obtain carbohydrates from the plants and provide nitrogen compounds to the plants by fixing nitrogen in the air. Therefore, by promoting the symbiosis of rhizobia, that is, by promoting the formation of root nodules in plants in which rhizobia coexist, the growth of the plants can be promoted.

Patent Literature 1 discloses that nucleobases such as inosine, guanosine, uridine, inosinic acid, guanylic acid, uridylic acid, hypoxanthine, guanine, and uracil promote root nodule formation in plants. Patent Literature 1 also discloses that by applying these nucleobases to soil or plants, root nodule formation of leguminous plants can be promoted, the growth of leguminous plants can be improved, and the amounts of nitrogen fertilizers used can be reduced.

In addition, Non Patent Literature 1 teaches that the expression of THI1 gene and TH1C gene, which are thiamine biosynthesis genes, was enhanced by inoculation with rhizobia and root nodules was small in a thi1 mutant, and discloses that thiamine biosynthesis by the thiamine biosynthesis genes promotes nodulation.

### CITATION LIST

### Non Patent Literature

Non Patent Literature 1: Plant Physiology, November 2016, Vol. 172, pp. 2033-2043

### Patent Literature

Patent Literature 1: JP Patent Publication (Kokai) No. 2011-132211 A

### SUMMARY OF INVENTION

### Technical Problem

However, even if the nucleobases disclosed in Patent Literature 1 and the thiamine disclosed in Non Patent Literature 1 promote root nodule formation, the mechanism of the root nodule formation process was unknown, and it was completely unknown what substance promotes root nodule formation.

Accordingly, it is an object of the present invention to provide a symbiosis-promoting agent capable of promoting root nodule formation, a symbiosis-promoting method, and a plant cultivation method in view of the above-described circumstances.

### Solution to Problem

In order to achieve the above-described object, the present inventors made intensive studies and as a result found that hydroxycinnamic acids promote root nodule formation. This has led to the completion of the present invention.

The present invention encompasses the following.
(1) A symbiosis-promoting agent comprising a cinnamic acid compound represented by the following formula:

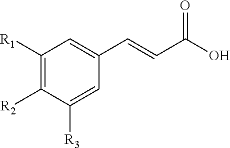

(where R₁, R₂, and R₃ may be the same or different and R₁, R₂, and R₃ are each independently a hydroxyl group (OH group), a hydrogen atom (H), an alkoxy group (OX), or a prenyl group (X is an alkyl group having 1 to 5 carbon atoms)).
(2) The symbiosis-promoting agent according to (1), wherein the cinnamic acid compound is a hydroxycinnamic acid compound in which at least one of R₁, R₂, and R₃ in the formula is a hydroxyl group.
(3) The symbiosis-promoting agent according to (1), wherein the cinnamic acid compound is at least one selected from cinnamic acid, p-coumaric acid, ferulic acid, sinapinic acid, and caffeic acid.
(4) A symbiosis-promoting method, comprising allowing a cinnamic acid compound represented by the following formula to act on plant roots:

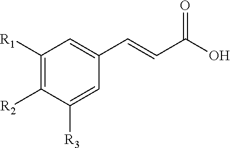

(where R₁, R₂, and R₃ may be the same or different and R₁, R₂, and R₃ are each independently a hydroxyl group (OH group), a hydrogen atom (H), an alkoxy group (OX), or a prenyl group (X is an alkyl group having 1 to 5 carbon atoms)).
(5) The symbiosis-promoting method according to (4), wherein the cinnamic acid compound is a hydroxycinnamic acid compound in which at least one of R₁, R₂, and R₃ in the formula is a hydroxyl group.
(6) The symbiosis-promoting method according to (4), wherein the cinnamic acid compound is at least one selected from cinnamic acid, p-coumaric acid, ferulic acid, sinapinic acid, and caffeic acid.
(7) A plant production method, comprising allowing a cinnamic acid compound represented by the following formula to act on plant roots:

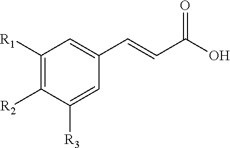

(where R₁, R₂, and R₃ may be the same or different and R₁, R₂, and R₃ are each independently a hydroxyl group (OH group), a hydrogen atom (11), an alkoxy group (OX), or a prenyl group (X is an alkyl group having 1 to 5 carbon atoms)).
(8) The plant production method according to (7), wherein the cinnamic acid compound is a hydroxycinnamic acid compound in which at least one of R₁, R₂, and R₃ in the formula is a hydroxyl group.
(9) The plant production method according to (7), wherein the cinnamic acid compound is at least one selected from cinnamic acid, p-coumaric acid, ferulic acid, sinapinic acid, and caffeic acid.

The present description encompasses the contents of the disclosure in JP Patent Application No. 2020-190053 which serves as the basis of the priority of the present application.

### Advantageous Effects of Invention

The symbiosis-promoting agent and the symbiosis-promoting method according to the present invention can promote root nodule formation by rhizobia. In addition, since the plant cultivation method according to the present invention comprises promoting root nodule formation by a cinnamic acid compound, a plant growth-promoting effect is sufficiently exhibited, and a plant with promoted growth can be produced.

### BRIEF DESCRIPTION OF DRAWINGS

FIG. 1 is a characteristic diagram showing the results of measuring the effect of promoting root nodule formation (number of root nodules, number of infection threads) by caffeic acid.

FIG. 2 is a characteristic diagram showing the results of measuring the effect of promoting root nodule formation (number of root nodules, number of infection threads) by ferulic acid.

FIG. 3 is a characteristic diagram showing the results of measuring the effect of promoting root nodule formation (number of root nodules, number of infection threads) by sinapinic acid.

FIG. 4 is a characteristic diagram showing the results of measuring the effect of increasing biomass production (aerial part length, underground part length, number of leaves) and the effect of promoting root nodule formation (number of root nodules) by ferulic acid (growing for one week after addition) on the soybean,

FIG. 5 is a characteristic diagram showing the results of measuring the effect of increasing biomass production (aerial part length, underground part length, number of leaves) and the effect of promoting root nodule formation (number of root nodules) by ferulic acid (growing for two weeks after addition) on the soybean.

### DESCRIPTION OF EMBODIMENTS

The present invention will be described in detail below.

### <Symbiosis-Promoting Agent>

The symbiosis-promoting agent according to the present invention (hereinafter simply referred to as “symbiosis-promoting agent”) contains a cinnamic acid compound represented by the following formula as an active ingredient:

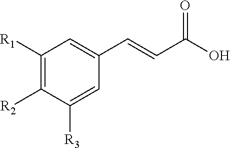

(where R₁, R₂, and R₃ may be the same or different and R₁, R₂, and R₃ are each independently a hydroxyl group (OH group), a hydrogen atom (H), an alkoxy group (OX), or a prenyl group (X is an alkyl group having 1 to 5 carbon atoms)).

The cinnamic acid compounds represented by the above formula encompass cinnamic acid in which all of R₁, R₂, and R₃ are hydrogen atoms (1H) and hydroxycinnamic acids in which at least one of R₁, R₂, and R₃ is a hydroxyl group (OH). Here, hydroxycinnamic acid is a polyphenol having a C₆-C₃ skeleton. The hydroxycinnamic acid represented by the above formula has a basic structure in which one carboxy group is bound to an aromatic ring via two carbon atoms.

The hydroxycinnamic acid represented by the above formula has three substituents R₁, R₂, and R₃ at the meta-position and para-position with respect to the Carboxy group bound to the aromatic ring. At least one of the three substituents R₁, R₂, and R₃ possessed by the hydroxycinnamic acid is a hydroxyl group (OH group, phenolic hydroxyl group). The three substituents R₁, R₂, and R₃ of the hydroxycinnamic acid may be independently the same or different.

The three substituents R₁, R₂, and R₃ in the above formula are any of a hydroxyl group (OH group), a hydrogen atom (H), an alkoxy group (OX), and a prenyl group. X in the alkoxy group (OX) represents an alkyl group having 1 to 5 carbon atoms. Examples of alkyl groups having 1 to 5 carbon atoms include methyl group, ethyl group, n-propyl group, i-propyl group, n-butyl group, sec-butyl group, tert-butyl group, neopentyl group, isopentyl group, sec-pentyl, 3-pentyl, and tert-pentyl.

The substituent R₂ in the above formula is preferably a phenolic hydroxyl group (OH group). When the substituent R₂ in the hydroxycinnamic acid is a phenolic hydroxyl group (OH group), the phenolic hydroxyl group (OH group) forms a conjugated system with a carboxy group bound to aromatic ring via two carbon atoms to become electronically stable.

Here, the hydroxycinnarnic acids represented by the above formula are meant to encompass hydroxycinnamate. Examples of hydroxycinnamate include acid addition salts, metal salts, ammonium salts, organic ammonium salts, organic amine addition salts, and amino acid addition salts. Examples of acid addition salts include: inorganic acid salts such as hydrochloride, sulfate, nitrate, and phosphate; and organic acid salts such as acetate, maleate, fumarate, citrate, malate, lactate, α-ketoglutarate, gluconate, and caprylate. Examples of metal salts include: alkali metal salts such as sodium salts and potassium salts; alkaline earth metal salts such as magnesium salts and calcium salts; aluminum salts, and zinc salts. Examples of organic ammonium salts include tetramethylammonium salts. Examples of organic amine addition salts include monoethanolamine salts, diethanolamine salts, triethanolamine salts, morpholine salts, and piperidine salts. Examples of amino acid addition salts include glycine salts, phenylalanine salts, lysine salts, aspartic acid salts, and glutamic acid salts.

More specific examples of the hydroxycinnarnic acids represented by the above formula include cinnamic acid, ferulic acid, sinapinic acid, p-coumaric acid, caffeic acid, isoferulic acid, and artepillin C. in particular, the hydroxycinnamic acid represented by the above formula is preferably at least one selected from the group consisting of p-coumaric acid, ferulic acid, sinapinic acid, and caffeic acid. The hydroxycinnamic acid represented by the above formula is more preferably at least one selected from the group consisting of ferulic acid, sinapinic acid, and caffeic acid.

The hydroxycinnamic acid used as an active ingredient of the symbiosis-promoting agent can be one type of the hydroxycinnamic acids represented by the above formula; however, a plurality of types of hydroxycinnamic acids may also be used as active ingredients. Specifically, the hydroxycinnamic acid contained in the symbiosis-promoting agent may be a mixed acid of ferulic acid and sinapinic acid, a mixed acid of ferulic acid and caffeic acid, a mixed acid of sinapinic acid and caffeic acid, or a mixed acid of ferulic acid, sinapinic acid, and caffeic acid. In these mixed acids, the composition ratio of each hydroxycinnamic acid can be appropriately adjusted.

Here, ferulic acid (3-methoxy-4-hydroxycinnamic acid) has the following structural formula, in which R₁, R₂, and R₃ in the above formula are a methoxy group (OCH₃), a phenolic hydroxyl group (OH group), and a hydrogen atom (H), respectively.

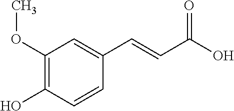

In addition, sinapinic acid has the following structural formula, in which R₁, R₂, and R₃ in the above formula are a methoxy group (OCH₃), a phenolic hydroxyl group (OH group), and a methoxy group (OCH₃), respectively.

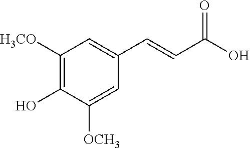 [SMILES: COc1cc(/C=C/C(=O)O)cc(OC)c1O]

Further, caffeic acid has the following structural formula, in which R₁, R₂, and R₃ in the above formula are a phenolic hydroxyl group (OH group), a phenolic hydroxyl group (OH group), and a hydrogen atom (H), respectively.

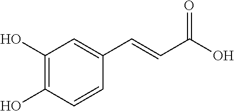 [SMILES: O=C(O)/C=C/c1ccc(O)c(O)c1]

Although no specific structural formula is shown for p-coumaric acid, R₁, R₂, and R₁ in the above formula are a hydrogen atom (H group), a phenolic hydroxyl group (01-1 group), and a hydrogen atom (1H), respectively.

The concentration of a cinnamic compound containing cinnamic acid and hydroxycinnamic acid as described above in the symbiosis-promoting agent is not particularly limited. However, it can be, for example, 1 μM to 1 mM, which is preferably 10 μM to 500 μM, more preferably 10 μM to 300 μM, still more preferably 10 PM to 100 μM.

### <Rhizobia>

A rhizobium that forms root nodules is not limited as long as it can coexist with plants to form root nodules, and the nodule-formation-promoting agent according to the present invention promotes root nodule formation. Examples of rhizobia include bacteria belonging to genera Rhizobium, Bradyrhizobium, Sinorhizobium, and Mesorhizobium, More specific examples thereof include Rhizobium leguminosarum, Rhizobium tropici, Sinorhizobium meliloti, Sinorhizobium fredii, Bradyrhizobium japonicum, Bradyrhizobium elkani, Mesorhizobium loti, and Mesorhizobium huakuii.

### <Plant Cultivation Method>

By using the symbiosis-promoting agent described above, it is possible to promote root nodule formation by a rhizobium in a plant to be cultivated. Accordingly, it is possible to increase the biomass of the plant. The phrase “using the symbiosis-promoting agent” described herein means an embodiment in which the above-described symbiosis-promoting agent is supplied to soil such that the symbiosis-promoting agent is allowed to act on a rhizobium contained in the soil and an embodiment in which a rhizobium is supplied together with the above-described symbiosis-promoting agent to soil.

In any of these embodiments, the above-described symbiosis-promoting agent acts on plant roots such that root nodule formation is promoted. This makes it possible to increase the biomass of the plant to be cultivated. The plant to be cultivated is not limited as long as a rhizobium can coexist therewith. Such plants may be monocotyledonous or dicotyledonous and edible or non-edible. Specific examples of such plants include leguminous plants.

Examples of leguminous plants include soybeans, adzuki beans, broad beans, peas, peanuts, cowpeas, lupin, clover, and alfalfa.

A method for supplying the symbiosis-promoting agent to soil is not particularly limited. The symbiosis-promoting agent can be supplied by, for example, methods such as spraying, mixing, embedding, chemical injection, and chemical watering into the soil. When the symbiosis-promoting agent is supplied to the soil, it may be applied to a part of the soil where the plant is grown or to the entire surface thereof. Specific examples of a place where the symbiosis-promoting agent or a microbial material is applied include planting holes or their vicinity, planting rows or their vicinity, between hills, the entire surface of the culture soil, the entire surface of the soil, nursery boxes, nursery trays, nursery pots, and nursery beds.

The symbiosis-promoting agent may be applied to the soil before or after sowing or planting plants. Examples of the application period include before sowing, during sowing, the period until budding after sowing, the budding period, the breeding period, during transplanting seedlings, during cutting or herbaceous cutting, the growing period after settled planting (e.g., before flowering, during flowering, after flowering, immediately before or during heading), and the start of fruit coloring. At that time, the symbiosis-promoting agent may be applied to the soil only once or a plurality of times. From the viewpoint of sufficiently obtaining the effect of promoting plant growth while reducing the application amount as much as possible, the symbiosis-promoting agent is applied preferably at the early growth stage of the plant (specifically, the period from budding to flowering or before heading) or earlier, more preferably at the nursery stage or earlier.

### EXAMPLES

The present invention will be described in more detail below using Examples, but the technical scope of the present invention is not limited to the following Examples.

### Example 1

In this Example, the effect of promoting root nodule formation by caffeic acid was verified.

First, caffeic acid (Caffeic Acid, CAS RN: 331-39-5 C0002, Tokyo Chemical Industry Co., Ltd.) was dissolved in ethanol, thereby preparing a 100 mM stock solution. Next, about 300 mL of vermiculite was added to pots, entirely moistened with water, and then sterilized in an autoclave. Miyakogusa (Lotus japonicus) three to four days after sowing was transplanted into the pots, and 50 mL of B&D liquid medium (Broughton and Dilworth, 1971) containing a DsRED gene-bearing rhizobium (OD600=0.1 mL; 1 mL) and caffeic acid (final concentration: 10 μM or 100 μM) was added.

Plants inoculated with the rhizobium were grown in an incubator (26° C.; 16 hr light/8 hr dark). The plants were dug up from the soil after one week and two weeks, and the number of infection threads and the number of root nodules were counted under a fluorescent stereomicroscope.

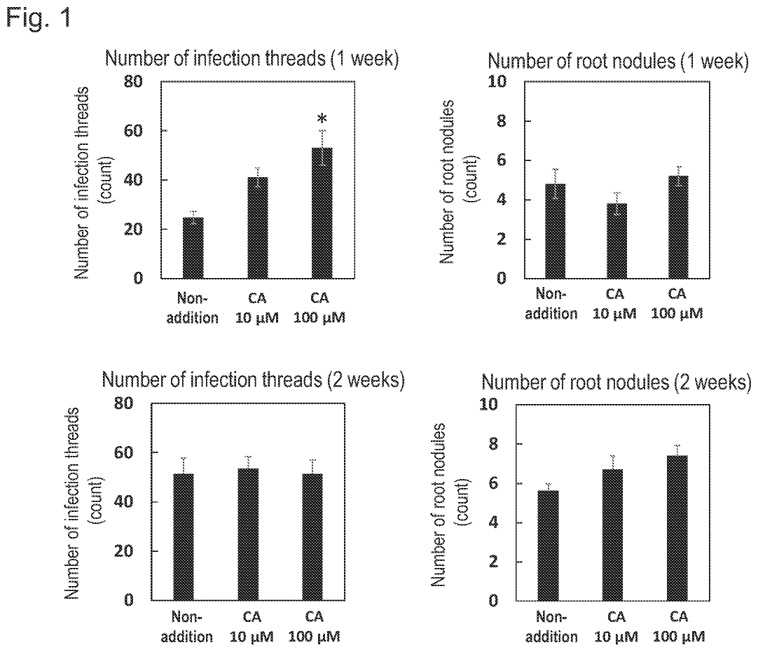

The measurement results are shown in FIG. 1. In FIG. 1, error bars in the graphs represent standard errors, and asterisks (*) indicate data with a significant difference (Dunnett method: p<0.05; n=10) compared to Non-addition (control). As shown in FIG. 1, the addition of caffeic acid caused a significant increase in the number of infection threads after one week and a tendency for the number of root nodules to increase after two weeks.

From the results of this Example, it was found that caffeic acid, which is a cinnamic acid compound, has an effect of promoting root nodule formation on rhizobia. Thus, using caffeic acid as a root nodule-formation-promoting agent is expected to increase the biomass production of root nodule-forming plants.

### Example 2

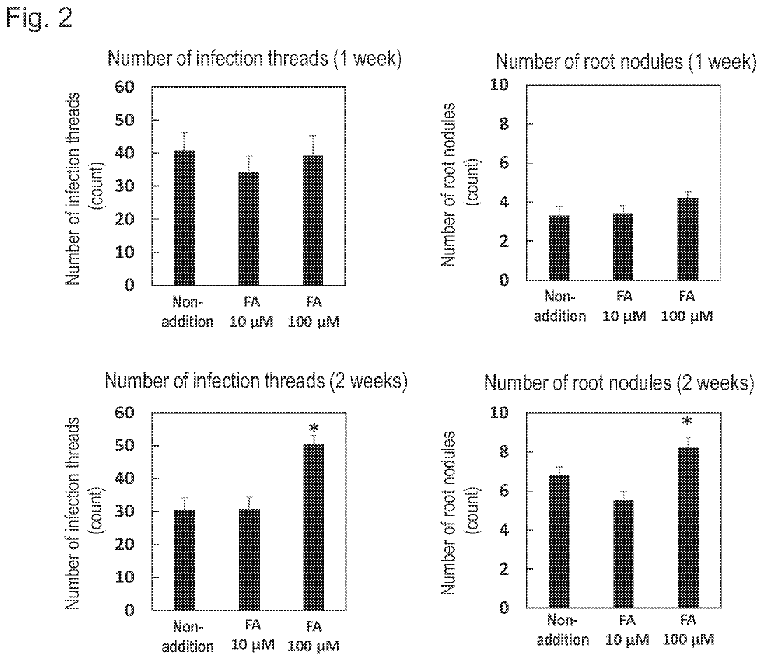

In this Example, the effect of promoting root nodule formation by ferulic acid was verified. In this Example, the number of infection threads and the number of root nodules were counted in the same manner as in Example 1, except that ferulic acid (trans-Ferulic Acid, CAS RN: 537-98-4 H-0267, Tokyo Chemical Industry Co., Ltd.) was used instead of caffeic acid. The measurement results are shown in FIG. 2. As shown in FIG. 2, the addition of ferulic acid caused significant increases in the number of infection threads and the number of root nodules after two weeks.

From the results of this Example, it was found that ferulic acid, which is a cinnamic acid compound, has an effect of promoting root nodule formation on rhizobia. Thus, using ferulic acid as a root nodule-formation-promoting agent is expected to increase the biomass production of root nodule-forming plants.

### Example 3

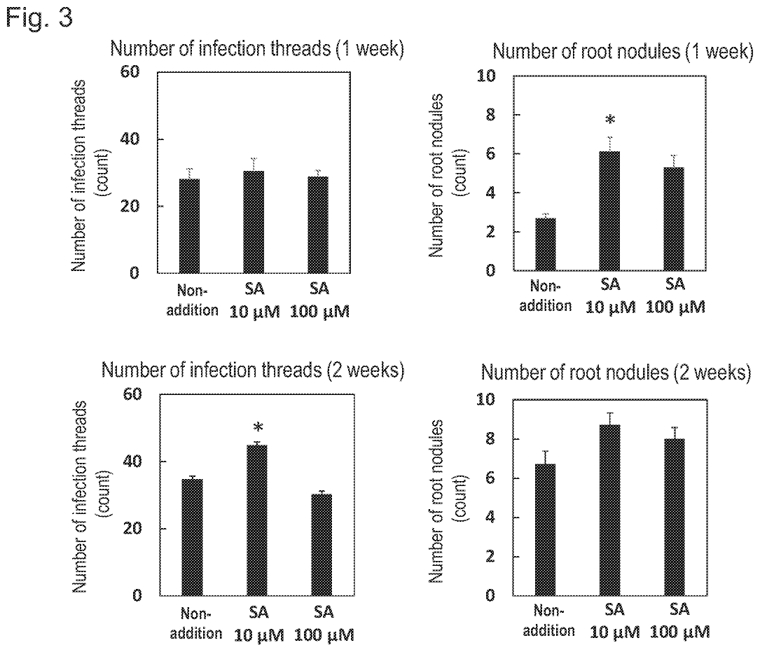

In this Example, the effect of promoting root nodule formation by sinapinic acid was verified. In this Example, the number of infection threads and the number of root nodules were counted in the same manner as in Example 1, except that sinapinic acid (3,5-Dimethoxy-4-hydroxycinnamic Acid [Matrix for MALDI-TOF/MS], CAS RN:530-59-6 D2932, Tokyo Chemical Industry Co., Ltd.) was used instead of caffeic acid. The measurement results are shown in FIG. 3. As shown in FIG. 3, the addition of sinapinic acid caused a significant increase in the number of root nodules after one week and a significant increase in the number of infection threads after two weeks.

From the results of this Example, it was found that sinapinic acid, which is a cinnamic acid compound, has an effect of promoting root nodule formation on rhizobia. Thus, using sinapinic acid as a root nodule-formation-promoting agent is expected to increase the biomass production of root nodule-forming plants.

### Example 4

In this Example, ferulic acid was used to verify the effect of increasing biomass production on soybeans.

In this Example, first, seeds of the soybean (Glycine max cv. Enrei) were placed on wet Kimwipes® for three to four days to germinate and then transferred to pots containing vermiculite (about 1.1 L) and grown for about three days in a plant incubator (28° C.; 16 hr light/8 hr dark). Next, a non-addition plot was prepared by adding rhizobia (Bradyrhizobium japonicum; OD600=0.1; 1 mL) to 50 ml of B&D medium. An addition plot was prepared by adding ferulic acid (final concentration: 100 μM) to 50 mL of B&D medium containing rhizobia. They were inoculated into the individual soybean plants. Soybean plants grown in the incubator for one week and two weeks were dug up from the pots, and their growth (aerial part length, underground part length, number of leaves) and number of root nodules were measured.

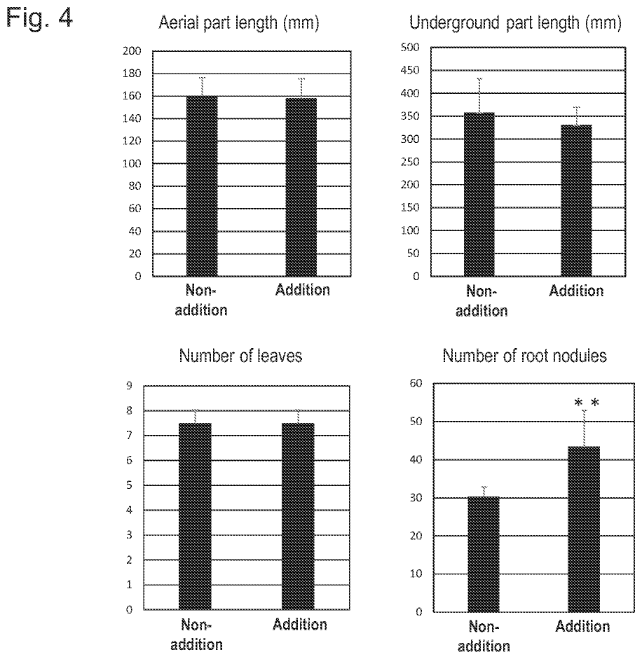

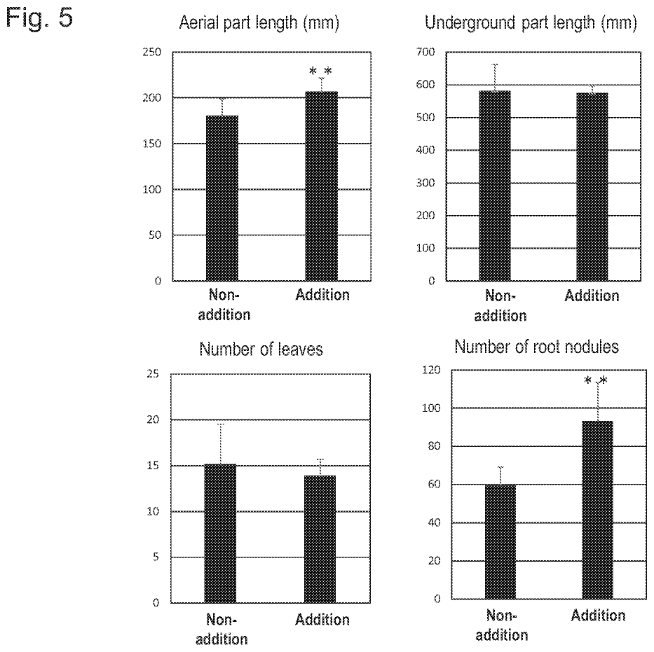

FIG. 4 shows the results of measuring soybean plants grown for one week, and FIG. 5 shows the results of measuring soybean plants grown for two weeks. In FIGS. 4 and 5, error bars in the graphs represent standard errors, and asterisks (**) indicate data with a significant difference (Dunnett method: p<0.01; n=8) compared to Non-addition (control).

As shown in FIG. 4, it was found that the number of root nodules significantly increased in soybean plants grown for one week with the addition of ferulic acid. Moreover, as shown in FIG. 5, it was found that, in addition to the number of root nodules, the aerial part length significantly increased in the soybean plants grown for two weeks with the addition of ferulic acid. From the results, it was found that ferulic acid, which is a cinnamic acid compound, has the effect of promoting root nodule formation on soybeans and also has the effect of increasing biomass production.

All publications, patents, and patent applications cited in the present description are incorporated herein by reference in their entirety.

1. A symbiosis-promoting agent comprising a cinnamic acid compound represented by the following formula: 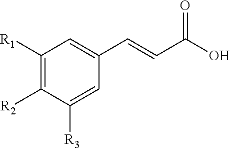
(where R₁, R₂, and R₃ may be the same or different and R₁, R₂, and R₃ are each independently a hydroxyl group (OH group), a hydrogen atom (H), an alkoxy group (OX), or a prenyl group (X is an alkyl group having 1 to 5 carbon atoms)).

2. The symbiosis-promoting agent according to claim 1, wherein the cinnamic acid compound is a hydroxycinnamic acid compound in which at least one of R₁, R₂, and R₃ in the formula is a hydroxyl group.

3. The symbiosis-promoting agent according to claim 1, wherein the cinnamic acid compound is at least one selected from cinnamic acid, p-coumaric acid, ferulic acid, sinapinic acid, and caffeic acid.

4. A symbiosis-promoting method, comprising allowing a cinnamic acid compound represented by the following formula to act on plant roots: 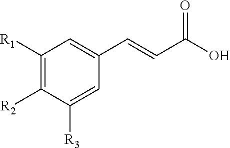
(where R₁, R₂, and R₃ may be the same or different and R₁, R₂, and R₃ are each independently a hydroxyl group (OH group), a hydrogen atom (H), an alkoxy group (OX), or a prenyl group (X is an alkyl group having 1 to 5 carbon atoms)).

5. The symbiosis-promoting method according to claim 4, wherein the cinnamic acid compound is a hydroxycinnamic acid compound in which at least one of R₁, R₂, and R₃ in the formula is a hydroxyl group.

6. The symbiosis-promoting method according to claim 4, wherein the cinnamic acid compound is at least one selected from cinnamic acid, p-coumaric acid, ferulic acid, sinapinic acid, and caffeic acid.

7. A plant production method, comprising allowing a cinnamic acid compound represented by the following formula to act on plant roots: 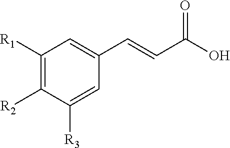
(where R₁, R₂, and R₃ may be the same or different and R₁, R₂, and R₃ are each independently a hydroxyl group (OH group), a hydrogen atom (H), an alkoxy group (OX), or a prenyl group (X is an alkyl group having 1 to 5 carbon atoms)).

8. The plant production method according to claim 7, wherein the cinnamic acid compound is a hydroxycinnamic acid compound in which at least one of R₁, R₂, and R₃ in the formula is a hydroxyl group.

9. The plant production method according to claim 7, wherein the cinnamic acid compound is at least one selected from cinnamic acid, p-coumaric acid, ferulic acid, sinapinic acid, and caffeic acid.
